(19) United States
(12) Patent Application Publication
(10) Pub. No.: US 2010/0197950 A1
(43) Pub. Date: Aug. 5, 2010

(54) PROCESS FOR PREPARING PHENOXY ACETIC ACID DERIVATIVES

(76) Inventors: Kaare Gyberg Rasmussen, Copenhagen (DK); Signe Maria Christensen, Copenhagen (DK); Rikke Eva Humble, Copenhagen (DK)

Correspondence Address: High Point Pharmaceuticals, LLC, 4170 Mendenhall Oaks Parkway, High Point, NC, 27265, US

(21) Appl. No.: 12/303,747

(22) PCT Filed: Jun. 6, 2007
(86) PCT No.: PCT/EP07/55568

§ 371 Date: Mar. 25, 2010

(30) Foreign Application Priority Data
Jun. 8, 2006 (EP) 06115124.7

(51) Int. Cl. C07C 309/67; C07C 25/28; C07C 17/363; C07C 303/28; C07C 51/377; C07C 67/32

(52) U.S. Cl. 558/55; 570/184; 570/200; 562/468; 560/61

(57) ABSTRACT

The preparation of an intermediate, a process for the preparation thereof and the process of preparing [4-[3,3-Bis-(4-bromo-phenyl)-allyloxy]-2-methyl-phenoxy]-acetic acid using this intermediate are described.

### FIELD OF THE INVENTION

This invention relates to the art of synthetic organic chemistry. More specifically, the invention relates to the preparation of a useful intermediate, a process for the preparation thereof and the process of preparing [4-[3,3-Bis-(4-bromo-phenyl)-allyloxy]-2-methyl-phenoxy]-acetic acid using this intermediate.

### BACKGROUND OF THE INVENTION

[4-[3,3-Bis-(4-bromo-phenyl)-allyloxy]-2-methyl-phenoxy]-acetic acid has been identified as a partial PPARS agonist (or “Selective PPARS Modulator” (SPPARδM)) with full efficacy on fatty acid (FFA) oxidation in vitro and plasma lipid correction in vivo.

Coronary artery disease (CAD) is the major cause of death in Type 2 diabetic and metabolic syndrome patients (i.e., patients that fall within the ‘deadly quartet’ category of impaired glucose tolerance, insulin resistance, hypertriglyceridaemia and/or obesity).

The hypolipidaemic fibrates and antidiabetic thiazolidinediones separately display moderately effective triglyceride-lowering activities, although they are neither potent nor efficacious enough to be a single therapy of choice for the dyslipidaemia often observed in Type 2 diabetic or metabolic syndrome patients. The thiazolidinediones also potently lower circulating glucose levels of Type 2 diabetic animal models and humans. However, the fibrate class of compounds are without beneficial effects on glycaemia. Studies on the molecular actions of these compounds indicate that thiazolidinediones and fibrates exert their action by activating distinct transcription factors of the peroxisome proliferator activated receptor (PPAR) family, resulting in increased and decreased expression of specific enzymes and apolipoproteins respectively, both key-players in regulation of plasma triglyceride content.

PPAR-δ activation was initially reported not to be involved in modulation of glucose or triglyceride levels. (Berger et al., J. Biol. Chem. 1999, 274, 6718-6725). Later it was shown that PPAR-δ activation leads to increased levels of HDL cholesterol in dbIdb mice (Leibowitz et al., FEBS letters 2000, 473, 333-336). Further, a PPAR-δ agonist when dosed to insulin-resistant middle-aged obese rhesus monkeys caused a dramitic dose-dependent rise in serum HDL cholesterol while lowering the levels of small dense LDL, fasting triglycerides and fasting insulin (Oliver et al., PNAS 2001, 98, 5306-5311). The same paper also showed that PPAR-δ activation increased the reverse cholesterol transporter ATP-binding cassette A1 and induced apolipoprotein A1-specific cholesterol efflux. The involvement of PPAR-δ in fatty acid oxidation in muscles was further substantiated in PPAR-a knock-out mice. Muoio et al. (J. Biol. Chem. 2002, 277, 26089-26097) showed that the high levels of PPAR-δ in skeletal muscle can compensate for deficiency in PPAR-α. Taken together these observations suggest that PPAR-δ activation is useful in the treatment and prevention of cardiovascular diseases and conditions including atherosclerosis, hypertriglyceridemia, and mixed dyslipidaemia (WO 01/00603).

A number of PPAR-δ compounds have been reported to be useful in the treatment of hyperglycemia, hyperlipidemia and hypercholesterolemia (WO 02/59098, WO 01/603, WO 01/25181, WO 02/14291, WO 01/79197, WO 99/4815, WO 97/28149, WO 98/27974, WO 97/28115, WO 97/27857, WO 97/28137, WO 97/27847 WO 2004093879, WO 2004092117, WO 2004080947, WO 2004080943, WO 2004073606, WO 2004063166, WO 2004063165, WO 2003072100, WO 2004060871, WO 2004005253, WO 2003097607, WO 2003035603, WO 2004000315, WO 2004000762, WO 2003074495, WO 2002070011, WO 2003084916, US 20040209936, WO 2003074050, WO 2003074051, WO 2003074052, JP 2003171275, WO 2003033493, WO 2003016291, WO 2002076957, WO 2002046154, WO 2002014291, WO 2001079197, WO 2003024395, WO 2002059098, WO 2002062774, WO 2002050048, WO 2002028434, WO 2001000603, WO 2001060807, WO 9728149, WO 2001034200, WO 9904815, WO 200125226, WO 2005097098; WO 2005097762, WO 2005097763).

Glucose lowering as a single approach does not overcome the macrovascular complications associated with Type 2 diabetes and metabolic syndrome. Novel treatments of Type 2 diabetes and metabolic syndrome must therefore aim at lowering both the overt hypertriglyceridaemia associated with these syndromes as well as alleviation of hyperglycaemia. This indicates that research for compounds displaying various degree of PPAR-δ activation should lead to the discovery of efficacious triglyceride and/or cholesterol and/or glucose lowering drugs that have great potential in the treatment of diseases such as type 2 diabetes, dyslipidemia, syndrome X (including the metabolic syndrome, i.e., impaired glucose tolerance, insulin resistance, hypertrigyceridaemia and/or obesity), cardiovascular diseases (including atherosclerosis) and hypercholesteremia.

Procedures for preparing phenoxy acetic acid derivatives are e.g. described in WO 2004037776, WO 2005105735, EP 334 596 and in Mogensen, et al; “Design and synthesis of novel PPAR α/γ/δ triple activators using a known PPARα/γ dual activator as structural template.”; Bioorg. Med. Chem. Lett. 2003, 13, 257-260. In McKillop, et al; “Functional group oxidation using sodium perborate”; Tetrahedron, 1987, Vol. 43, no. 8, 1753-1758, McKillop, et al; “Sodium perborate and sodium percarbonate: further applications in organic synthesis”; J. Chem. Soc., Perkin Trans., 2000; 471-476; and in Bandgar, et al; “Facile and selective deprotection of aryl acetates using sodium perborate under mild and neutral conditions”; New J. Chem., 2002, 26, 1273-1276, various procedures for preparing and deprotecting aryl acetates are described.

It has now been found that these procedures may be optimized under conditions amenable for production to obtain [4-[3,3-Bis-(4-bromo-phenyl)-allyloxy]-2-methyl-phenoxy]-acetic acid in a satisfactory yield, and at the same time by a more economically and environmentally acceptable process, than the previously disclosed processes.

### SUMMARY OF THE INVENTION

In one aspect of the invention, a compound of the general formula I

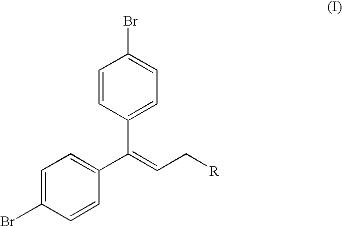

wherein R is selected from the group consisting of halogen and OSO₂R¹, wherein R¹ is C₁₋₆-alkyl or C₁₋₆-alkyl-aryl, is provided.

In a further aspect of the invention, a process for preparing a compound of formula I

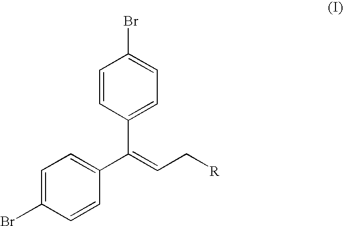

wherein R is selected from the group consisting of halogen and OSO₂R¹, wherein R¹ is C₁₋₆-alkyl or C₁₋₆-alkyl-aryl,
comprising the steps of
(a) reducing a 3,3-bis-(4-bromphenyl)-acrylic acid ester with formula III, wherein R³ is selected from the group consisting of C₁₋₆-alkyl and aryl-C₁₋₆-alkyl

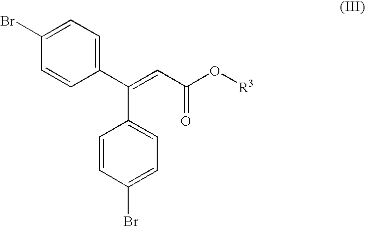

to an 3,3-bis-(4-bromophenyl)-prop-2-en-ol with formula IV

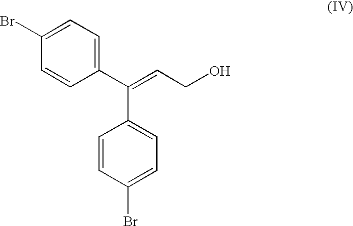

and
(b) reacting the compound of formula IV with
a halogenating agent to form the compound of formula I, wherein R is halogen or
a reagent selected from the group consisting of SO₂R⁴ and R⁵SO₂R⁴, wherein R⁴ is halogen and R⁵ is selected from the group consisting of C₁₋₆-alkyl and C₁₋₆-alkyl-aryl, to form the compound of formula I, wherein R is OSO₂R¹,
   is provided.

In a further aspect of the invention, a process for preparing a compound of formula II

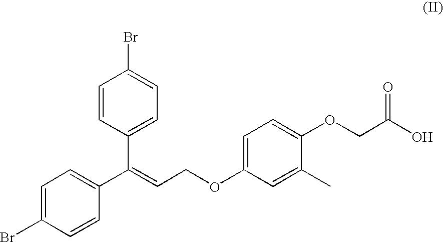

comprising the steps of
(a1) reacting a compound of formula I

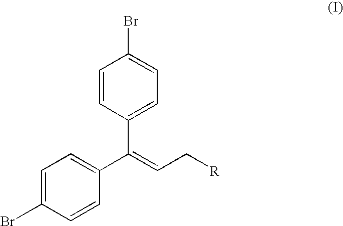

wherein R is selected from the group consisting of halogen and OSO₂R¹, wherein R¹ is C₁₋₆-alkyl or C₁₋₆-alkyl-aryl,
with a compound of formula V, wherein R² is C₁₋₆-alkyl

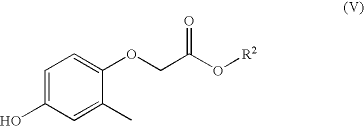

by an aliphatic nucleophilic substitution to form the ester of the compound of formula II and
(b1) hydrolysing said ester, optionally without isolation, to obtain the compound with formula II, is provided.

In a further aspect of the invention, a process for preparing a compound of formula V, wherein R² is C₁₋₆-alkyl

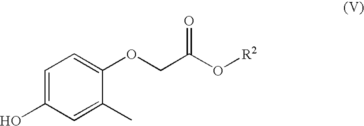

comprising the steps of
(a2) oxidising the compound with formula VI, wherein R⁶ is a protecting group and R⁷ is selected from the group consisting of a C₁₋₆-alkyl and aryl-C₁₋₆-alkyl, by a Baeyer-Villiger oxidation using an oxidizing agent

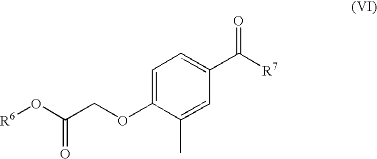

to a compound with formula VII

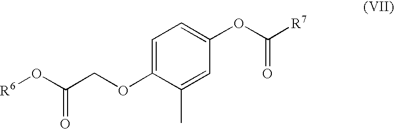

and
(b2) optionally without isolation of the compound of formula VII, deprotecting the compound of formula VII with a stoichiometric amount of sodium perborate hydrated to obtain the compound of formula V, and
(c2) isolating the compound of formula V by precipitation in an aqueous solution, is provided.

### DEFINITIONS

As used herein the term “halogenating agent” refers to halogenic acids or other reagents capable of converting alcohols to halides. Illustrative halogenating agents include HCl, HBr, HI, SOCl₂, SO₂Cl₂ PCl₃, POCl₃, PCl₅ and the like.

The term “halogen” or “halo” means fluorine, chlorine, bromine or iodine.

The term “hydroxy” shall mean the radical —OH.

The term “C₁₋₆-alkyl” as used herein represents a saturated, branched or straight hydro-carbon group having from 1 to 6 carbon atoms, e.g. C₁₋₃-alkyl, C₁₋₆-alkyl, C₂₋₆-alkyl, C₃₋₆-alkyl, and the like. Representative examples are methyl, ethyl, propyl (e.g. prop-1-yl, prop-2-yl (or iso-propyl)), butyl (e.g. 2-methylprop-2-yl (or tert-butyl), but-1-yl, but-2-yl), pentyl (e.g. pent-1-yl, pent-2-yl, pent-3-yl), 2-methylbut-1-yl, 3-methylbut-1-yl, hexyl (e.g. hex-1-yl), and the like.

The term “aryl” as used herein is intended to include monocyclic, bicyclic or polycyclic carbocyclic aromatic rings. Representative examples are phenyl, naphthyl (e.g. naphth-1-yl, naphth-2-yl), anthryl (e.g. anthr-1-yl, anthr-9-yl), phenanthryl (e.g. phenanthr-1-yl, phenanthr-9-yl), and the like.

The term “optionally substituted” as used herein means that the groups in question are either unsubstituted or substituted with one or more of the substituents specified. When the group(s) in question are substituted with more than one substituent the substituents may be the same or different.

Certain of the defined terms may occur more than once in the structural formulae, and upon such occurrence each term shall be defined independently of the other.

Certain of the defined terms may occur in combinations, and it is to be understood that the first mentioned radical is a substituent on the subsequently mentioned radical, where the point of substitution, i.e. the point of attachment to another part of the molecule, is on the last mentioned of the radicals. Such combinations of terms include for example:

The term “aryl-C₁₋₆-alkyl” as used herein refers to the radical aryl-C₁₋₆-alkyl-. Representative examples are benzyl, phenethyl (e.g. 1-phenylethyl, 2-phenylethyl), phenylpropyl (e.g. 1-phenylpropyl, 2-phenylpropyl), and the like.

The term “C₁₋₆-alkyl-aryl” as used herein refers to the radical C₁₋₆-alkyl-aryl-. Representative examples are methyl phenyl, and the like.

“C₁₋₆-alkylsulfonyl” as used herein refers to the radical C₁₋₆-alkyl-S(═O)₂—. Representative examples are methylsulfonyl, ethylsulfonyl, propylsulfonyl, butylsulfonyl, pentylsulfonyl, hexylsulfonyl, and the like.

“arylsulfonyl” as used herein refers to the radical aryl-S(═O)₂—. Representative examples are phenylsulfonyl, (4-methylphenyl)sulfonyl, (4-chlorophenyl)sulfonyl, naphthylsulfonyl, and the like.

Representative protecting groups include, for example, C₁₋₆-alkyl and substituted C₁₋₆-alkyl, including methyl, ethyl, isopropyl, cyclopropyl, methoxymethyl, methylthiomethyl, tert-butyl-thiomethyl, (phenyldimethylsilyl)methoxymethyl, benzyloxymethyl, p-methoxy-benzyloxy-methyl, tert-butoxy-methyl, ethoxyethyl, 1-(2-chloroethoxy)ethyl, 2,2,2-trichloroethoxy-methyl, and 2-(trimethylsilyl)ethyl; phenyl and substituted phenyl groups such as p-chloro-phenyl, p-methoxyphenyl, and 2,4-dinitrophenyl; benzyl groups; alkylsilyl groups such as trimethyl- triethyl- and triisopropylsilyl; mixed alkylsilyl groups such as dimethylisopropyl-silyl, and diethylisopropylsilyl; acyl protecting groups such as those of the general formula COC₁₋₆-alkyl or COAr; and esters of the general formula CO₂C₁₋₆-alkyl, or CO₂Ar, where Ar is phenyl or substituted phenyl as described above.

Representative deprotecting agents include, for example, lithium, sodium or potassium alkoxide of hydroxyl-C₁₋₆-alkyl and sodium perborate (with 1 or 4 crystal water).

Representative reducing agents include, for example, diisobutyl aluminiumhydride, lithium borohydride, lithium triethylborohydride, lithium aluminium hydride, sodium bis-[2-methoxy-ethoxy]-aluminium hydride and alane.

The term “suitable solvent” refers to any solvent, or mixture of solvents, that sufficiently solubilizes the reactants to afford a medium within which to effect the desired reaction.

Suitable solvents include methanol, acetic acid, methylene chloride, chloroform, 1,2-dichloro-ethane, diethyl ether, acetonitrile, ethyl acetate, 1,3-dimethyl-2-imidazolidinone, 1,4-dioxane, tetrahydrofuran, toluene, chlorobenzene, N-methylpyrrolidinone (NMP), dimethyl formamide (DMF), dimethyl acetamide (DMA), toluene, xylene, halophenyl solvents such as chlorobenzene, etheral solvents such as glyme, diglyme and ethyleneglycol diether ether, mixtures thereof, and the like. Toluene is a preferred solvent.

The term “aliphatic nucleophilic substitution” refers to an organic reaction in which a nucleophile with an electron pair forms a bond to the substrate, and the leaving group in the substrate comes away with an electron pair. A phase transfer reaction is an example of an aliphatic nucleophilic substitution reaction. An aliphatic nucleophilic substitution can be carried out in a biphasic solvent system by means of phase transfer catalysis (PTC). In the case of an alcohol reacting with a substrate having a halide leaving group the reaction is referred to as a Williamson reaction.

The term Baeyer-Villiger oxidation refers to an organic reaction in which a ketone is oxidized to an ester by treatment with an oxidizing agent. Agents typically used to carry out this rearrangement are e.g. meta-chloroperoxybenzoic acid (m-CPBA), peroxyacetic acid, anhydrous hydrogen peroxide, urea-hydrogen peroxide complex, peroxytrifluoroacetic acid and sodium perborate hydrated. In one aspect of the invention, the agent is sodium perborate hydrated.

### DESCRIPTION OF THE INVENTION

In one aspect, the invention provides a compound of the general formula I

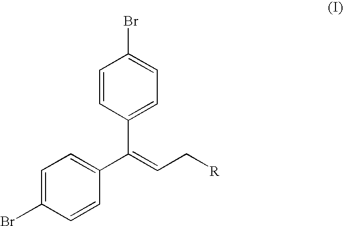

wherein R is selected from the group consisting of halogen and OSO₂R¹, wherein R¹ is C₁₋₆-alkyl or C₁₋₅-alkyl-aryl.

In one aspect of the invention, R is halogen. In a further aspect of the invention, R is selected from the group consisting of chlorine, bromine and iodine. In yet a further aspect of the invention, R is chlorine.

In one aspect of the invention, R¹ is methyl. In a further aspect of the invention, R¹ is methyl phenyl.

Intermediate compounds of the formula I of the invention are for example:
3,3-Bis(4-bromophenyl)-3-chloro-1-propene,
3,3-Bis(4-bromophenyl)-3-bromo-1-propene,
3,3-Bis(4-bromophenyl)-3-iodo-1-propene,
methanesulfonic acid 3,3-bis-(4-bromo-phenyl)-allyl ester, and
toluene-4-sulfonic acid 3,3-bis-(4-bromo-phenyl)-allyl ester.

The compound of formula I is particular useful as an intermediate in the process for preparing a compound of formula II

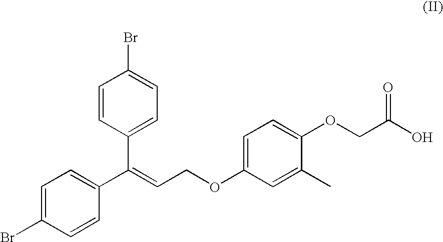

In a further aspect of the Invention, a process for preparing a compound of formula I

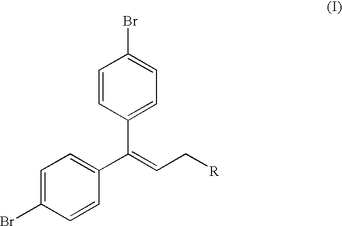

wherein R is selected from the group consisting of halogen and OSO₂R¹, wherein R¹ is C₁₋₆-alkyl or C₁₋₆-alkyl-aryl,
comprising the steps of
(a) reducing a 3,3-bis-(4-bromphenyl)-acrylic acid ester with formula III, wherein R³ is selected from the group consisting of C₁₋₆-alkyl and aryl-C₁₋₆-alkyl

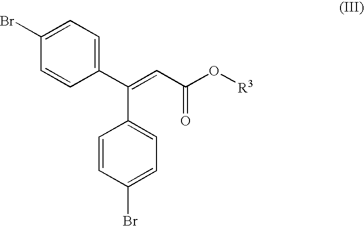

to an 3,3-bis-(4-bromophenyl)-prop-2-en-ol with formula IV

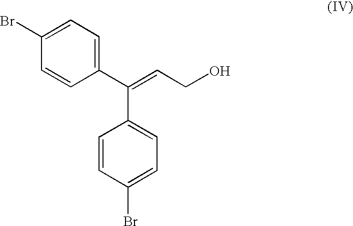

and
(b) reacting the compound of formula IV with
a halogenating agent to form the compound of formula I, wherein R is halogen, or
a reagent selected from the group consisting of SO₂R⁴ and R⁵SO₂R⁴, wherein R⁴ is halogen and R⁵ is selected from the group consisting of C₁₋₆-alkyl and C₁₋₆-alkyl-aryl, to form the compound of formula I, wherein R is OSO₂R¹,
   is provided.

In one aspect of the invention, R is halogen. In a further aspect of the invention, R is selected from the group consisting of chlorine, bromine and iodine. In yet a further aspect of the invention, R is chlorine.

In one aspect of the invention, R³ is C₁₋₆-alkyl, such as ethyl. In another aspect of the invention, R³ is aryl-C₁₋₆-alkyl, such as benzyl.

Intermediate compounds of the formula III of the invention are for example:
3,3-Bis-(4-bromophenyl)-acrylic acid methyl ester,
3,3-Bis-(4-bromophenyl)-acrylic acid ethyl ester,
3,3-Bis-(4-bromophenyl)-acrylic acid propyl ester,
3,3-Bis-(4-bromophenyl)-acrylic acid isopropyl ester,
3,3-Bis-(4-bromophenyl)-acrylic acid n-butyl ester,
3,3-Bis-(4-bromophenyl)-acrylic acid sec-butyl ester,
3,3-Bis-(4-bromophenyl)-acrylic acid tert-butyl ester, and
3,3-Bis-(4-bromophenyl)-acrylic acid benzyl ester.

As an example, the above process according to the invention for preparing the compound of formula I, wherein R is chlorine, is illustrated in Scheme 1 below:

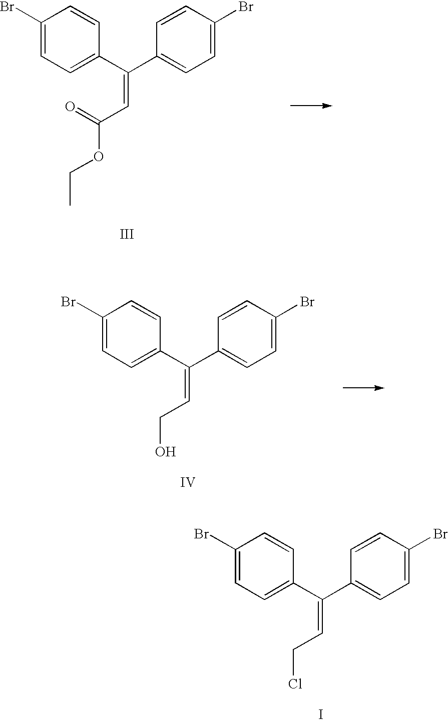

The process shown in Scheme 1 can be performed in a first step by contacting a compound of formula III dissolved in a suitable solvent with a reduction agent such as diisobutyl aluminumhydride (DIBAL) to effect a reduction to an alcohol of formula IV.

The above process shown in Scheme 1 can further in a second step proceed by reacting the obtained compound of formula IV after aqueous work-up and phase separation with a halogenating agent, or an reagent such as C₁₋₆alkyl-arylsulphonylchloride or C₁₋₆alkyl-sulphonylchloride to give the compound of formula I.

In one aspect of the invention, the compound of formula IV is treated with the halogenating agent SO(R⁴)₂ wherein R⁴ is halogen, such as chlorine, bromine and iodine. In a further aspect of the invention the halogenating agent is thionylchloride (SOCl₂).

In a further aspect of the invention, the compound of formula IV is treated with a reagent selected from the group consisting of SO₂R⁴ wherein R⁴ is halogen and R⁵SO₂R⁴, wherein R⁴ is halogen and R⁵ is C₁₋₆-alkyl or C₁₋₆-alkyl-aryl. In yet a further aspect of the invention, R⁴ is chlorine. In yet another aspect of the invention, R⁵ is methyl. In yet another aspect of the invention, R⁵ is methyl phenyl.

It has been found in one aspect of the invention, that the temperature during step (a) and/or step (b) is in the interval of 5-80° C. In a further aspect of the invention, the temperature is in the interval of 10-50° C. In yet a further aspect of the invention, the temperature is 50° C. In yet a further aspect of the invention, the temperature is 40° C. In yet a further aspect of the invention, the temperature is in the interval of 15-30° C.

In another aspect of the invention, the solvent in step (a) and/or step (b) is selected from the group consisting of toluene, tetrahydrofuran (THF), N-methylpyrrolidinone (NMP), dimethyl formamide (DMF), and dimethyl acetamide (DMA). In a further aspect of the invention, the solvent in step (a) and/or step (b) is selected from the group consisting of toluene, NMP, DMF, and DMA. In yet a further aspect of the invention, the solvent is toluene in step (a).

In yet a further aspect of the invention, a compound of formula III is dissolved in toluene and added to a solution of a reduction agent such as DIBAL in toluene.

In yet a further aspect of the invention, the solvent is toluene in step (b).

In yet a further aspect of the invention, the solvent is toluene in both step (a) and step (b).

In yet a further aspect of the invention, the compound obtained in step (a) is telescoped into step (b) after an aqueous wash.

The time required to effect the overall transformation illustrated in Scheme 1 will be dependant upon e.g. the temperature at which the reaction is run, the concentration of the substrate, and the excess of reagent. Therefore the progress of the reactions should be monitored via conventional techniques, e.g. HPLC, to determine when the reactions are substantially complete. Monitoring the progress of chemical reactions is well within the capability of the skilled person.

Due to the high yield, the product solution of the compound of formula I may be used as is in subsequent reactions or the product may be isolated by conventional methods for solvent removal and/or crystallisation.

As described above the compound of formula I is useful for the preparation of the compound of formula II.

The invention thus relates in a further aspect to a process for preparing a compound of

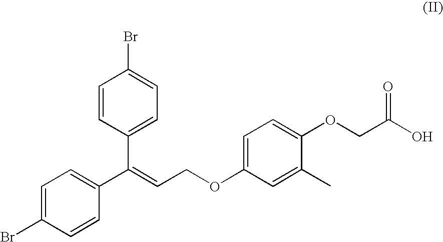

comprising the steps of
(a1) reacting a compound of formula I

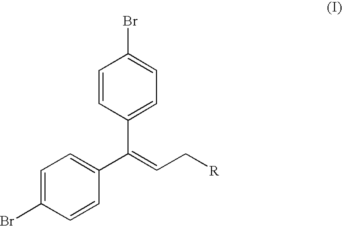

wherein R is selected from the group consisting of a halogen or OSO₂R¹, wherein R¹C₁₋₆-alkyl or C₁₋₆-alkyl-aryl
with a compound of formula V, wherein R² is C₁₋₆-alkyl

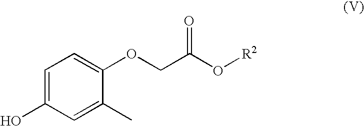

by an aliphatic nucleophilic substitution to form the ester of the compound of formula II and
(b1) hydrolysing said ester, optionally without isolation, to obtain the compound with formula II.

In one aspect of the invention, the reaction in step (a1) is an aliphatic nucleophilic substitution.

In another aspect of the invention, R² is selected from the group consisting of methyl and ethyl. In a further aspect of the invention, R² is methyl.

As an example, the above process according to the invention for preparing the compound of formula II is illustrated in Scheme 2 below:

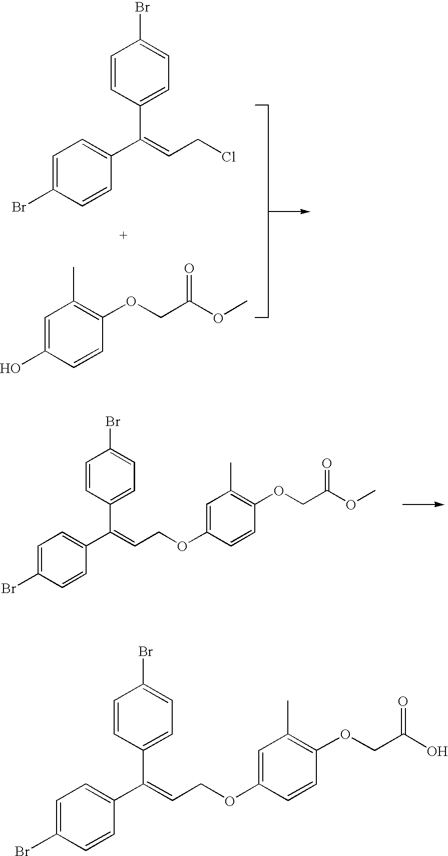

The above process illustrated in Scheme 2 can be performed by contacting a compound of formula I dissolved in a suitable solvent with a compound of formula V in the presence of a base and optionally a catalyst, such as sodium iodide, potassium iodide and similar iodide salts to effect an alkylation to an ester of formula II by nucleophilic substitution.

Basic hydrolysis of the ester intermediates results in the desired compound of formula II. This hydrolysis may be performed with or without isolation of the ester of the compound of formula II.

In one aspect of the invention, the solvent used in step (a1) and/or step (b1) is selected from the group consisting of toluene, THF, acetonitrile, methyl ethyl ketone (MEK), NMP, DMF, and DMA. In yet a further aspect of the invention, the solvent is acetonitrile in step (a1). In yet a further aspect of the invention, the solvent is acetonitrile in step (a1) and step (b1). In another aspect of the invention, the first base used in step (a1) for nucleophilic substitution and/or the second base used in step (b1) for hydrolysing said ester is selected from the group consisting of sodium hydroxide, potassium hydroxide, sodium hydrogencarbonate, potassium hydrogencarbonate, sodium carbonate, potassium carbonate, cesium carbonate, triethylamine, n-methylmorpholine, and diisopropylethylamine.

In yet a further aspect of the invention, the base is cesium carbonate in step (a1).

In yet a further aspect of the invention, the treatment with cesium carbonate in step (a1) is followed by treatment with sodium hydroxide or potassium hydroxide in step (b1).

In yet a further aspect of the invention, the compound obtained in step (a1) is telescoped into step (b1).

In one aspect of the invention, the aliphatic nucleophilic substitution is performed by phase transfer.

When performing the reaction by the use of phase transfer catalysis (PTC) this involves contacting a compound of formula I (which is soluble in the organic layer) dissolved in an appropriate solvent such as dimethyl glycol and a compound of formula V (a nucleophile), which is dissolved in an aqueous layer. The substrate and the anion are then brought together by a catalyst such as quaternary ions, tertiary amine or crown ether, which transports the anion into the organic phase where reaction can take place.

The time required to effect the overall transformation will be dependant upon the temperature at which the reaction is run, the concentration of the substrates, the solvent, the base and the optionally added catalyst. As described above the progress of the reactions should be monitored via conventional techniques, e.g. HPLC, to determine when the reactions are substantially complete. Monitoring the progress of chemical reactions is well within the capability of the skilled person.

In a further aspect the invention relates to a process for preparing a compound of formula V, wherein R² is C₁₋₆-alkyl

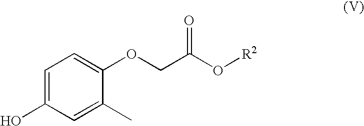

comprising the steps of
(a2) oxidising the compound with formula VI, wherein R⁶ is a protecting group and R⁷ is selected from the group consisting of a C₁₋₆-alkyl and aryl-C₁₋₆-alkyl, by a Baeyer-Villiger oxidation using an oxidizing agent

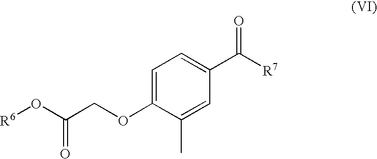

to a compound with formula VII

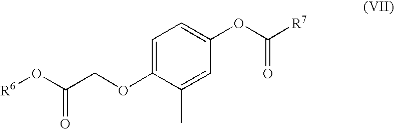

and
(b2) optionally without isolation of the compound of formula VII, deprotecting the compound of formula VII with a stoichiometric amount of sodium perborate hydrated to obtain the compound of formula V, and
(c2) isolating the compound of formula V by precipitation in an aqueous solution, is provided.

In one aspect of the invention, the compound of formula VII is isolated as a crystalline compound.

In another aspect of the invention, the aqueous solution is an aqueous buffer solution.

In one aspect of the invention, the compound of formula VI is a compound where R⁷ is C₁₋₆alkyl. In a further aspect of the invention, the compound of formula VI is a compound where R⁷ is methyl. In a further aspect of the invention, the compound of formula VI is a compound where R⁶ is methyl.

Examples of intermediate compounds of formula VI are:
(4-Acetyl-2-methyl-phenoxy)-acetic acid methyl ester,
(4-Acetyl-2-methyl-phenoxy)-acetic acid ethyl ester,
(4-Acetyl-2-methyl-phenoxy)-acetic acid propyl ester,
(4-Acetyl-2-methyl-phenoxy)-acetic acid isopropyl ester,
(4-Acetyl-2-methyl-phenoxy)-acetic acid n-butyl ester,
(4-Acetyl-2-methyl-phenoxy)-acetic acid sec-butyl ester,
(4-Acetyl-2-methyl-phenoxy)-acetic acid tert-butyl ester, and
(4-Acetyl-2-methyl-phenoxy)-acetic acid benzyl ester.

Examples of intermediate compounds of formula VII are
(4-Acetoxy-2-methyl-phenoxy)-acetic acid methyl ester,
(4-Acetoxy-2-methyl-phenoxy)-acetic acid ethyl ester,
(4-Acetoxy-2-methyl-phenoxy)-acetic acid propyl ester,
(4-Acetoxy-2-methyl-phenoxy)-acetic acid isopropyl ester,
(4-Acetoxy-2-methyl-phenoxy)-acetic acid n-butyl ester,
(4-Acetoxy-2-methyl-phenoxy)-acetic acid sec-butyl ester,
(4-Acetoxy-2-methyl-phenoxy)-acetic acid tert-butyl ester, and
(4-Acetoxy-2-methyl-phenoxy)-acetic acid benzyl ester.

Examples of intermediate compounds of formula V are
(4-Hydroxy-2-methyl-phenoxy)-acetic acid methyl ester,
(4-Hydroxy-2-methyl-phenoxy)-acetic acid ethyl ester,
(4-Hydroxy-2-methyl-phenoxy)-acetic acid propyl ester,
(4-Hydroxy-2-methyl-phenoxy)-acetic acid isopropyl ester,
(4-Hydroxy-2-methyl-phenoxy)-acetic acid n-butyl ester,
(4-Hydroxy-2-methyl-phenoxy)-acetic acid sec-butyl ester,
(4-Hydroxy-2-methyl-phenoxy)-acetic acid tert-butyl ester, and
(4-Hydroxy-2-methyl-phenoxy)-acetic acid benzyl ester.

As an example, the above process according to the invention for preparing the compound of formula V is illustrated in Scheme 3 below:

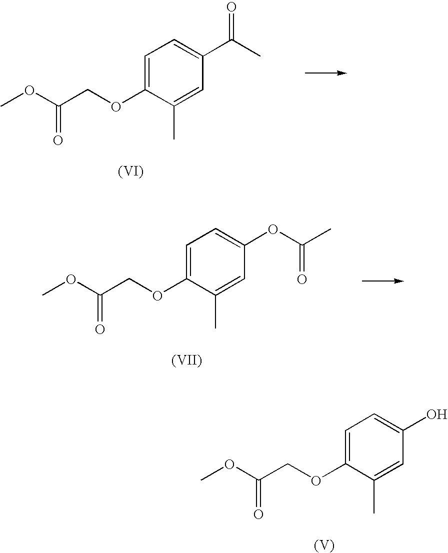

The instant process shown in Scheme 3 can be performed by oxidising a compound of formula VI dissolved in a suitable solvent by a Baeyer-Villiger oxidation using an oxidising agent to give a compound of formula VII, followed by a deprotection with a stoichiometric amount of sodium perborate hydrated in alcohol to obtain the compound of formula V and isolation of formula V by precipitation in an aqueous solution.

It has been found that a suitable temperature during step (a2) and/or step (b2) is in the interval of from 18 to 80° C. In a further aspect of the invention, the temperature is in the interval of from 18° C. to 65° C. In another aspect of the invention, the solvent is selected from the group consisting of acetic acid, formic acid, trifluoro acetic acid, methanol, toluene, and DMF. In yet a further aspect of the invention, the solvent is acetic acid in step (a2). In yet a further aspect of the invention, the solvent in step (b2) is a mixture of toluene and an alcohol or alcohol alone. In a further aspect of the invention, the alcohol is methanol. In yet a further aspect of the invention, the compound obtained in step (a2) is telescoped into step (b2) after an aqueous wash.

In a further aspect of the invention, the oxidizing agent used in the Baeyer-Villiger oxidation is selected from the group consisting of peroxoacids, such as meta-chloroperoxybenzoic acid (m-CPBA), peroxyacetic acid, peroxytrifluoroacetid acid, sodium perborate hydrated (such as sodium perborate monohydrate or sodium perborate tetrahydrate), urea-hydrogen peroxide complex, anhydrous hydrogen peroxide, peroxyacetic acid, or peroxytrifluoroacetic acid. In a further aspect of the invention, the oxidizing agent is sodium perborate hydrated (such as sodium perborate monohydrate or sodium perborate tetrahydrate) which is a stable, crystalline and easily handled oxidant. Sodium perborate hydrated is a useful reagent for the controlled Bayer-Villiger oxidation and is furthermore a cheap and non-toxic reagent which is safe to handle and without effluent of by-product problems. The reaction may easily be scaled up.

In a further aspect of the invention, the deprotection and the oxidising agent is the same and is sodium perborate hydrated.

The time required to effect the overall transformation will be dependant upon e.g the temperature at which the reaction is run and the concentration of the substrates. As described above the progress of the reactions should be monitored via conventional techniques, e.g. HPLC, to determine when the reactions are substantially complete. Monitoring the progress of chemical reactions is well within the capability of the skilled person.

Due to the high yield, the product solution may be used as is in subsequent reactions or the product may be isolated by conventional methods for solvent removal.

The features disclosed in the foregoing description may, both separately and in any combination thereof, be material for realising the invention in diverse forms thereof.

The following examples are illustrative only and are not intended to limit the scope of the invention in any way.

### EXAMPLES

### Experimental Section

NMR data were recorded on a 400 MHz spectrometer, with solvent peak as internal reference value (DMSO: 39.86 for ¹³C and 2.50 for ¹H. TMS-peak was used in CDCl₃). 1-(4-Hydroxy-3-methyl-phenyl)-ethanone was acquired from Apollo Scientific, methyl bromo acetate from Merck, sodium perborate monohydrate from Aldrich, triethyl phosphonoacetate from Alfa Aesar, 4-4′-dibromo-benzophenone from DKSH. All solvents used were HPLC-grade. HPLC analysis was performed using a column from Merck (cat. # 1.50377), solvent: 90% aceto-nitrile with 0.1% H₃PO₄, column temperature: 35° C., flow: 0.9 mL/min, UV-detector: 210 nm.

### Example 1

### Ethyl 3,3-bis-(4-bromo-phenyl)-acrylate

Sodium (40.6 g, 1.765 mol) was dissolved in absolute ethanol (1 L). Triethyl phosphono-acetate (395 g, 1.765 mol) was added, and the resulting mixture was stirred at 60° C. for 15 minutes. 4,4′-Dibromobenzophenone (500 g, 1.471 mol) was added to the reaction mixture and the temperature rose to 75° C. Additional ethanol (1 L) was added and the reaction mixture was stirred overnight at 70° C. The reaction was filtered while hot, and the filtrate was subsequently cooled to 10° C. Ethyl-3,3-bis-(4-bromophenyl)-acrylate precipitated, the product was isolated by filtration and the filter cake was washed with ethanol (300 mL). Dried overnight at room temperature to give 540 g ethyl 3,3-bis-(4-bromo-phenyl)-acrylate (90% yield). 1H-NMR (CDCl3): δ 7.52 (2H, d, J=8.5 Hz), 7.46 (2H, d, 3 J=8.6 Hz), 7.14 (2H, d, J=8.5 Hz), 7.07 (2H, d, J=8.1 Hz), 6.34 (1H, s), 4.07 (2H, q, J=7 Hz, CH2), 1.15 (3H, t, J=7 Hz, CH3). 13C-NMR (CDCl3): δ 166.0, 154.5, 139.6, 137.6, 132.1, 131.7, 131.2, 130.1, 124.5, 123.1, 118.6, 60.7, 14.4.

### Example 2

### 3,3-Bis-(4-bromophenyl)-prop-2-en-1-ol

To a stirred solution of 1 M DIBAL-H in toluene (1620 g, 1.888 mol) cooled to 13° C. was added a solution of the compound of example 1 (352 g, 0.858 mol) in toluene (500 mL), while the temperature of reaction mixture was kept below 25° C. HPLC after 1 h showed residual starting material, and more 1 M DIBAL-H in toluene (50 mL) was added. The resulting mixture was stirred for additional 10 minutes. Reaction mixture was transferred to a cooled mixture of conc. HCl (700 mL) in water (1.4 L), while the temperature was kept below 27° C. Layers were separated and the aqueous layer was extracted with toluene (500 mL). The combined organic layer was washed with water (2×400 mL) and concentrated in vacuo. The residue was crystallized from acetonitrile (600 mL) to afford 293 g of 3,3-Bis-(4-bromo-phenyl)-prop-2-en-1-ol (93% yield). ¹H-NMR (CDCl₃): δ 7.51 (2H, d, J=8.6 Hz), 7.41 (2H, d, J=8.5 Hz), 7.09 (2H, d, 3 J=8.6 Hz), 7.02 (2H, d, J=8.5 Hz), 6.23 (1H, t, J=7 Hz, CH), 4.19 (2H, d, J=6.6 Hz, CH₂), 1.50 (1H, br s, OH). ¹³C-NMR (CDCl₃): δ 142.1, 140.3, 137.4, 131.6, 131.44, 131.37, 129.2, 128.5, 122.0, 60.5.

### Example 3

### 1,1-Bis-(4-bromo-phenyl)-3-chloropropene

To a stirred solution of 3,3-Bis-(4-bromophenyl)-prop-2-en-1-ol of example 2 (250 g, 679 mmol) in toluene (1 L) was slowly added thionyl chloride (89 g, 747 mmol). NOTE: reaction set up was connected to a scrubber to remove formed HCl(g) and SO₂. The temperature of the reaction mixture rose to 32° C., and the mixture was stirred at this temperature for 2 h.

HPLC showed full conversion of starting material. Water (300 mL) was added, stirred for 30 min and the layers were separated. Toluene-layer was washed with water (300 mL) and was subsequently poured into a vigorously stirred saturated NaHCO₃-solution (400 mL). Layers were separated and the org. layer was washed with water (300 mL) and concentrated in vacuo. The residue was crystallized from isopropanol (900 mL), filtered and dried to afford 203 g of 1,1-Bis-(4-bromo-phenyl)-3-chloropropene (77% yield). ¹H-NMR (CDCl₃): δ 7.55 (2H, d, J=8.6 Hz), 7.42 (2H, d, J=8.6 Hz), 7.10 (2H, d, J=5.0 Hz), 7.08 (2H, d, J=5.6 Hz), 6.23 (1H, t, J=8 Hz, CH), 4.08 (2H, d, J=8.1 Hz, CH₂). ¹³C-NMR (CDCl₃): δ 144.5, 140.1, 137.0, 132.2, 131.9, 131.7, 129.7, 125.0, 123.0, 122.9, 42.5.

### Example 4

### Methyl (4-acetyl-2-methyl-phenoxy)-acetate

1-(4-Hydroxy-3-methyl-phenyl)-ethanone (300 g, 1.998 mol) was dissolved in methyl-ethyl-ketone (MEK) (3 L), potassium carbonate (552 g, 3.99 mol) and methyl bromoacetate (204 mL, 2.20 mol) was added and the resulting mixture was stirred at room temperature overnight. HPLC showed residual starting material, so more methyl bromoacetate was added (18.5 mL, 0.2 mol) and the resulting mixture was stirred at room temperature over night. Filtration of the reaction mixture, washing of the filter cake with MEK (0.5 L), and concentration of the combined filtrate in vacuo afforded a solid residue, which was dissolved in isopropanol (400 mL) and allowed to crystallize while stirred at room temperature for 3 days. The precipitated product was filtered, washed twice with ice-cold isopropanol and dried in vacuum oven at 40° C. overnight to afford methyl (4-acetyl-2-methyl-phenoxy)-acetate as a white solid (397.73 g, 90% yield). HPLC purity: 99.7%. ¹H-NMR (DMSO): δ 7.79 (1H, s), 7.79 (1H, d, J=7.0 Hz), 6.96 (1H, d, J=9 Hz), 4.95 (2H, s, CH₂), 3.71 (3H, s, CH₃), 2.51 (3H, s, CH₃), 2.25 (3H, s, CH₃). ¹³C-NMR (DMSO, 100 MHz): δ 196.8, 169.3, 159.9, 131.1, 130.4, 128.6, 111.3, 65.2, 52.2, 26.7, 16.3.

### Example 5

### Methyl (4-acetoxy-2-methyl-phenoxy)-acetate

Methyl (4-acetyl-2 methylphenoxy)-acetate of example 4 (200.0 g, 0.90 mol) was dissolved in acetic acid (1.80 L) and heated to 45-50° C. To this stirred solution was added sodium perborate monohydrate (269.2 g, 2.697 mol, 3 equiv.) at such rate that the temperature of the reaction was held between 50-62° C. After complete addition of sodium perborate, the reaction mixture was stirred over night at 45-50° C. HPLC showed full conversion of starting material. Mechanical stirring was stopped and the mixture was decanted to leave inorganic salts in the glass reactor. The decanted solution was concentrated in vacuo, a total of 1.8 L acetic acid was distilled. The concentrated solution was added water (1.5 L) and toluene (1 L). The layers were separated and the toluene layer was tested for peroxides (2 mg/L). Sodium bisulfite Na₂S₂O₅ (53 g) was added to the toluene layer, and the suspension was stirred for 30 minutes. Toluene layer was washed with water (500 mL) and concentrated to dryness to afford an orange oil, which crystallized upon standing (198 g, 92% yield). The aqueous layer was extracted with toluene (500 mL) to afford an extra 3.1 g of product. The inorganic salts from the reaction mixture was stirred with toluene (500 mL) for 30 minutes, filtered and concentrated in vacuo to afford an extra 10 g of product. A total of 211 g of product was isolated (99% yield). HPLC purity: 91.7%. ¹H-NMR (DMSO, 400 MHz): δ 6.93 (1H, br s), 6.85 (2H, br s), 4.82 (2H, s, CH₂), 3.70 (3H, s, CH₃), 2.22 (3H, s, CH₃), 2.19 (3H, s, CH₃). ¹³C-NMR (DMSO, 100 MHz) δ 169.8, 169.7, 153.7, 144.4, 127.6, 124.1, 119.9, 112.4, 65.51, 52.15, 21.12, 16.27.

### Example 6

### Methyl 2-(4-hydroxy-2-methyl-phenoxy)-acetate

Methyl (4-acetoxy-2-methyl-phenoxy)-acetate of example 5 (194 g, 0.81 mol) was dissolved in methanol (1.20 L) and added sodium perborate monohydrate (81.3 g, 0.81 mol, 1 equiv.). The reaction mixture was stirred by mechanical stirring at room temperature until HPLC showed full conversion to desired product (typically 4 hours, but reaction can be stirred overnight at room temperature without problems). When HPLC of reaction mixture shows full conversion of starting material, the reaction mixture was filtered on a glass filter packed with Hyflo Super Cel Celite 545 (50 g). Filter was washed with methanol (100 mL). Water (5 L) was added to the clear filtrate under stirring. A white solid precipitated and the suspension was stirred for 1 hour. The product was isolated by filtration and dried in vacuum oven over night (122.7 g, 77 oh yield). HPLC purity: 98.0%. ¹H-NMR (DMSO, 400 MHz): δ 8.86 (1H, br s, OH), 6.65 (1H, d, J=8.5 Hz), 6.58 (1H, d, J=2.5 Hz), 6.49 (1H, dd, J=3 Hz, J=8.6 Hz), 4.66 (2H, s, CH₂), 3.68 (3H, s, CH₃), 2.13 (3H, s, CH₃) ¹³C-NMR (DMSO, 100 MHz): δ 170.0, 151.8, 149.1, 127.6, 117.9, 113.5, 112.8, 66.1, 52.0, 16.4

### Example 7

### Methyl [4-[3,3-bis-(4-bromo-phenyl)-allyloxy]-2-methyl-phenoxy]-acetate

1,1-Bis-(4-bromophenyl)-3-chloropropene of example 3, (140 g, 0.362 mol) and methyl 2-(4-hydroxy-2-methyl-phenoxy)-acetate of example 6, (71.1 g, 362 mmol) was dissolved in acetonitrile (2 L) by heating to 40° C. The mixture was cooled to 23° C., added cesium carbonate (260 g, 0.797 mmol) and stirred at room temperature for 3 days. TLC (eluent: CH₂Cl₂) showed full conversion of starting materials. Reaction mixture was partitioned between water (900 mL) and toluene (900 mL), layers were separated and the aqueous layer was extracted with toluene (300 mL). The combined organic layer was concentrated in vacuo. The residue was dissolved in toluene (400 mL), filtered and concentrated in vacuo to afford methyl [4-[3,3-bis-(4-bromo-phenyl)-allyloxy]-2-methyl-phenoxy]-acetate as a solid residue (205 g, 104%) which was used for the next step without further purification.

### Example 8

### [4-[3,3-Bis-(4-bromo-phenyl)-allyloxy]-2-methyl-phenoxy]-acetic acid

The ester of example 7 (255.3 g, 0.467 mol) was dissolved in 96% ethanol (3.5 L) by warming to 75° C. 11 N Sodium hydroxide (85 mL, 935 mmol) diluted with water (710 mL) was added and the resulting mixture was stirred at 67° C. for 30 min. HPLC showed full conversion of starting material. 12 N Hydrochloric acid (86 mL, 1.028 mol) diluted with water (940 mL) was added and the resulting mixture was cooled to 8° C. The precipitated product was isolated by filtration and the filter cake was washed with water (3×250 mL). The solid was dried over night in vacuum oven at 40° C. to afford 4-[3,3-Bis-(4-bromo-phenyl)-allyl-oxy]-2-methyl-phenoxy]-acetic acid (232.5 g, 93.5% yield). HPLC purity: 98.8%. ¹H-NMR (DMSO): δ 12.9 (1H, br s, COOH), 7.64 (2H, d, J=8.1 Hz), 7.53 (2H, d, J=8.6 Hz), 7.16 (2H, d, J=7.6 Hz), 7.14 (2H, d, J=8.5 Hz), 6.71 (1H, d, J=9.1 Hz), 6.70 (1H, br s), 6.62 (1H, dd, J=2.5 Hz, 3 J=9 Hz), 6.37 (1H, t, 3 J=6.6 Hz), 4.59 (2H, s), 4.46 (2H, d, J=6.5

Hz), 2.14 (3H, s). ¹³C-NMR (DMSO): δ 170.8, 152.4, 150.7, 142.5, 140.2, 137.4, 131.92, 131.90, 131.8, 129.7, 127.7, 126.0, 121.8, 121.7, 117.8, 112.7, 112.4, 66.02, 65.7, 16.52.

### Example 9

### Methyl [4-[3,3-bis-(4-bromo-phenyl)-allyloxy]-2-methyl-phenoxy]-acetate

1,1-Bis-(4-bromophenyl)-3-chloropropene, (19.3 g, 50.0 mmol) and methyl 2-(4-hydroxy-2-methyl-phenoxy)-acetate (9.8 g, 50.0 mmol) was stirred with acetonitrile (290 mL). The mixture was added cesium carbonate (20 g, 61.4 mmol) and stirred at room temperature for 4 days. HPLC showed full conversion of starting materials. The reaction mixture was added water (80 mL) and a solid precipitated. The product Methyl [4-[3,3-bis-(4-bromo-phenyl)-allyloxy]-2-methyl-phenoxy]-acetate was isolated by filtration and washed with water (3×20 mL) and dried in vacuum oven over night (22.9 g, 84% yield). HPLC purity: 97.7%.

### Example 10

### [4-[3,3-Bis-(4-bromo-phenyl)-allyloxy]-2-methyl-phenoxy]-acetic acid

Methyl [4-[3,3-bis-(4-bromo-phenyl)-allyloxy]-2-methyl-phenoxy]-acetate (27.3 g, 50.0 mmol) was dissolved in acetonitrile (275 mL) by heating to 65° C. 11 N Sodium hydroxide (12.3 g, 100 mmol) diluted with water (75 mL) was added and the resulting mixture was stirred at 65° C. for 30 minutes. HPLC showed full conversion of starting material. Phosphoric acid (85%) (28.9 g, 250 mmol) diluted with water (33 mL) was added. Water (200 mL) was added and the mixture precipitated. The mixture was stirred at 20° C. over night. The precipitated product was isolated by filtration and the filter cake was washed with water (3×50 mL). The solid was dried over night in vacuum oven to afford 4-[3,3-Bis-(4-bromo-phenyl)-allyloxy]-2-methyl-phenoxy]-acetic acid (25.5 g, 96% yield). HPLC purity: 99.0%.

### Example 11

### [4-[3,3-Bis-(4-bromo-phenyl)-allyloxy]-2-methyl-phenoxy]-acetic acid

1,1-Bis-(4-bromophenyl)-3-chloropropene of example 3, (96.6 g, 0.250 mol) and methyl 2-(4-hydroxy-2-methyl-phenoxy)-acetate of example 6, (49.1 g, 250 mmol) was stirred with acetonitrile (500 mL). The mixture was added cesium carbonate (97.7 g, 300 mmol) and heated to 50° C. and stirred for 18 hours. HPLC showed full conversion of starting materials. 11 N Sodium hydroxide (55.5 mL, 611 mmol) diluted with water (250 mL) was added and the resulting mixture was stirred at 65° C. for 1 hour. HPLC showed full conversion of starting material. Phosphoric acid (85%) (84.3 mL, 1.25 mol) diluted with water (167 mL) was added and the mixture was cooled to 20° C. Water (325 mL) was added slowly and the mixture precipitated. The mixture was stirred at 20° C. over night. The precipitated product was isolated by filtration and the filter cake was washed with water (3×138 mL). The solid was dried over night in vacuum oven at 40° C. to afford 4-[3,3-Bis-(4-bromo-phenyl)-allyloxy]-2-methyl-phenoxy]-acetic acid (123.5 g, 93% yield). HPLC purity: 95.9%.

While the invention has been described and illustrated with reference to certain preferred embodiments thereof, those skilled in the art will appreciate that various changes, modifications, and substitutions can be made therein without departing from the spirit and scope of the present invention. Accordingly, the invention is not to be limited as by the appended claims.

The features disclosed in the foregoing description and/or in the claims may both separately and in any combination thereof be material for realising the invention in diverse forms thereof.

### PREFERRED FEATURES OF THE INVENTION

1. A compound of the general formula I

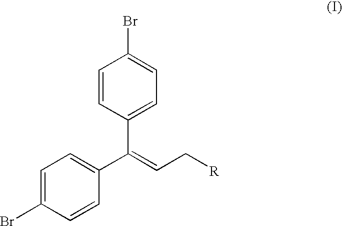

wherein R is selected from the group consisting of halogen and OSO₂R¹, wherein R¹ is C₁₋₆-alkyl or C₁₋₆-alkyl-aryl.

2. The compound according to clause 1, wherein R is halogen.

3. The compound according to clause 2, wherein R is selected from the group consisting of chlorine, bromine and iodine.

4. The compound according to clause 3, wherein R is chlorine.

5. The compound according to any one of the clauses 1-4, wherein R¹ is methyl.

6. The compound according to any one of the clauses 1-4, wherein R¹ is methyl phenyl.

7. The compound according to clause 1, which compound is selected from the group consisting of
3,3-Bis(4-bromophenyl)-3-chloro-1-propene,
3,3-Bis(4-bromophenyl)-3-bromo-1-propene,
3,3-Bis(4-bromophenyl)-3-iodo-1-propene,
methanesulfonic acid 3,3-bis-(4-bromo-phenyl)-allyl ester and
toluene-4-sulfonic acid 3,3-bis-(4-bromo-phenyl)-allyl ester.

8. A process for preparing a compound of formula I

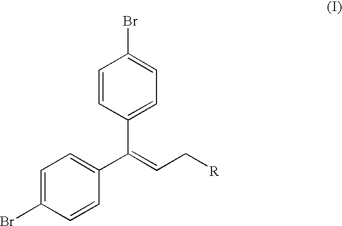

wherein R is selected from the group consisting of halogen and OSO₂R¹, wherein R¹ is C₁₋₆-alkyl or C₁₋₆-alkyl-aryl,
comprising the steps of
(a) reducing a 3,3-bis-(4-bromphenyl)-acrylic acid ester with formula III, wherein R³ is selected from the group consisting of C₁₋₆-alkyl and aryl-C₁₋₆-alkyl

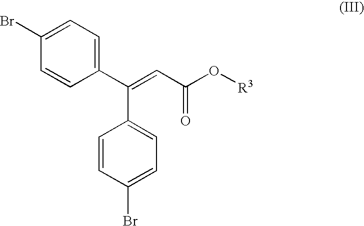

to an 3,3-bis-(4-bromophenyl)-prop-2-en-ol with formula IV

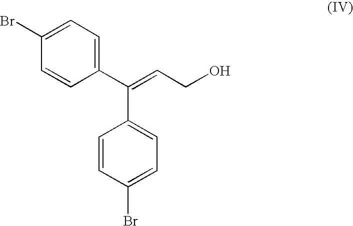

and
(b) reacting the compound of formula IV with
a halogenating agent to form the compound of formula I, wherein R is halogen, or
a reagent selected from the group consisting of SO₂R⁴ and R⁵SO₂R⁴, wherein R⁴ is halogen and R⁵ is selected from the group consisting of C₁₋₆-alkyl and C₁₋₆-alkyl-aryl, to form the compound of formula I, wherein R is OSO₂R¹.

9. Process according to clause 8, wherein the compound of formula I is a compound wherein R is halogen.

10. Process according to clause 9, wherein the compound of formula I is a compound wherein R is chlorine.

11. Process according to clause 8, wherein the compound of formula III is a compound wherein R³ is ethyl.

12. Process according to clause 8, wherein the compound of formula III is a compound selected from the group consisting of
3,3-Bis-(4-bromophenyl)-acrylic acid methyl ester,
3,3-Bis-(4-bromophenyl)-acrylic acid ethyl ester,
3,3-Bis-(4-bromophenyl)-acrylic acid propyl ester,
3,3-Bis-(4-bromophenyl)-acrylic acid isopropyl ester,
3,3-Bis-(4-bromophenyl)-acrylic acid n-butyl ester,
3,3-Bis-(4-bromophenyl)-acrylic acid sec-butyl ester,
3,3-Bis-(4-bromophenyl)-acrylic acid tert-butyl ester, and
3,3-Bis-(4-bromophenyl)-acrylic acid benzyl ester.

13. Process according to any one of the clauses 8-12, wherein the halogenating agent is SO(R⁴)₂ wherein R⁴ is halogen.

14. Process according to clause 13, wherein the halogenating agent is SO(R⁴), wherein R⁴ is chlorine, bromine and iodine.

15. Process according to clause 14, wherein the halogenating agent is SOCl₂.

16. Process according to any one of the clauses 8-12, wherein the reagent is selected from the group consisting of SO₂R⁴ and R⁵SO₂R⁴, wherein R⁴ is halogen and R⁵ is C₁₋₆-alkyl or C₁₋₆-alkyl-aryl.

17. Process according to clause 16, wherein R⁴ is chlorine.

18. Process according to any one of the clauses 16-17, wherein R⁵ is methyl.

19. Process according to any one of the clauses 16-17, wherein R⁵ is methyl phenyl.

20. Process according to any one of the clauses 8-19, wherein the temperature is in the interval of 5-80° C.

21. Process according to clause 20, wherein the temperature is in the interval of 10-50° C.

22. Process according to clause 20, wherein the temperature is in the interval of 15-30° C.

23. Process according to any one of the clauses 8-22, wherein the solvent in step (a) and/or step (b) is selected from the group consisting of toluene, N-methylpyrrolidinone (NMP), dimethyl formamide (DMF), and dimethyl acetamide (DMA).

24. Process according to any one of the clauses 8-23, wherein the solvent is toluene in step (a).

25. Process according to any one of the clauses 8-24, wherein the solvent is toluene in step (a) and step (b).

26. Process according to any one of the clauses 8-25, wherein the compound obtained in step (a) is telescoped into step (b) after an aqueous wash.

27. A process for preparing a compound of formula II

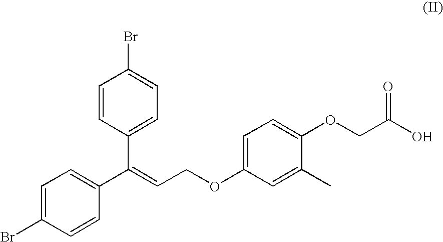

comprising the steps of
(a1) reacting a compound of formula I

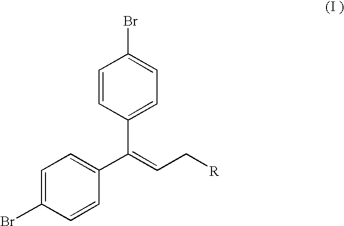

wherein R is selected from the group consisting of halogen and OSO₂R¹, wherein R¹ is C₁₋₆-alkyl or C₁₋₅-alkyl-aryl,
with a compound of formula V, wherein R² is C₁₋₆-alkyl

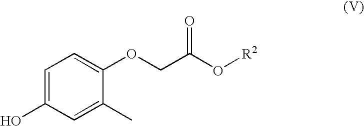

by an aliphatic nucleophilic substitution to form the ester of the compound of formula II and
(b1) hydrolysing said ester, optionally without isolation, to obtain the compound with formula II.

28. Process according to clause 27, wherein the compound of formula V is a compound where R² is selected from the group consisting of methyl and ethyl.

29. Process according to clause 28, wherein the compound of formula V is a compound where R² is selected from the group consisting of methyl.

30. Process according to any one of the clauses 27-29, wherein the compound of formula I is a compound, wherein R is halogen.

31. Process according to clause 30, wherein the compound of formula I is a compound, wherein R is selected from the group consisting of chlorine, bromine and iodine.

32. Process according to clause 31, wherein the compound of formula I is a compound, wherein R is selected from the group consisting of chlorine, bromine and iodine.

33. Process according to clause 32, wherein the compound of formula I is a compound, wherein R is chlorine.

34. Process according to any one of the clauses 27-33, wherein the aliphatic nucleophilic substitution is performed by phase transfer.

35. A process for preparing a compound of formula V, wherein R² is C₁₋₆-alkyl

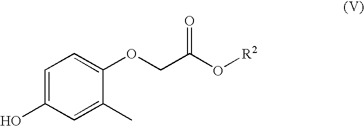

comprising the steps of
(a2) oxidising the compound with formula VI, wherein R⁶ is a protecting group and R⁷ is selected from the group consisting of a C₁₋₆-alkyl and aryl-C₁₋₆-alkyl, by a Baeyer-Villiger oxidation using an oxidizing agent

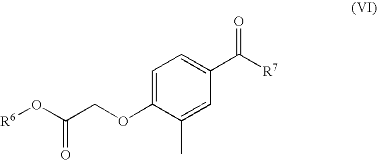

to a compound with formula VII

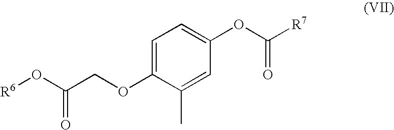

and
(b2) optionally without isolation of the compound of formula VII, deprotecting the compound of formula VII with a stoichiometric amount of sodium perborate hydrated to obtain the compound of formula V, and
(c2) isolating the compound of formula V by precipitation in an aqueous solution.

36. Process according to clause 35, wherein the compound of formula VI is a compound where R⁷ is C₁₋₆alkyl.

37. Process according to clause 36, wherein the compound of formula VI is a compound where R⁷ is methyl.

38. Process according to any one of the clauses 35-37, wherein the compound of formula VI is a compound where R⁶ is methyl.

39. Process according to any one of the clausems 35-38, wherein the oxidizing agent used in Baeyer-Villiger oxidation is selected from the group consisting of peroxoacids, such as meta-chloroperoxybenzoic acid (m-CPBA), sodium perborate hydrated (such as sodium perborate monohydrate or sodium perborate tetrahydrate), urea-hydrogen peroxide complex, anhydrous hydrogen peroxide, peroxyacetic acid, and peroxytrifluoroacetic acid.

40. Process according to clause 39, wherein the oxidizing agent is sodium perborate hydrated.

41. Process according to any one of the clauses 35-40, wherein the solvent in step (b2) is an alcohol or a mixture of an alcohol or toluene.

42. Process according to any one of the clauses 35-41, wherein the solvent in step (b2) is an alcohol.

1. A compound of the general formula I 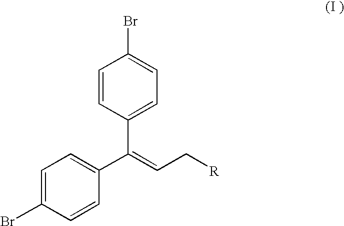
wherein R is selected from the group consisting of halogen and OSO₂R¹, wherein R¹ is C₁₋₆-alkyl or C₁₋₆-alkyl-aryl.

2. The compound according to claim 1, which compound is selected from the group consisting of
3,3-Bis(4-bromophenyl)-3-chloro-1-propene,
3,3-Bis(4-bromophenyl)-3-bromo-1-propene,
3,3-Bis(4-bromophenyl)-3-iodo-1-propene,
methanesulfonic acid 3,3-bis-(4-bromo-phenyl)-allyl ester and
toluene-4-sulfonic acid 3,3-bis-(4-bromo-phenyl)-allyl ester.

3. A process for preparing a compound of formula I 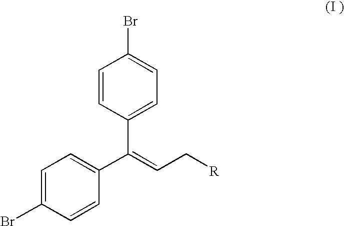
wherein R is selected from the group consisting of halogen and OSO₂R¹, wherein R¹ is C₁₋₆-alkyl or C₁₋₆-alkyl-aryl,
comprising the steps of
(a) reducing a 3,3-bis-(4-bromphenyl)-acrylic acid ester with formula III, wherein R³ is selected from the group consisting of C₁₋₆-alkyl and aryl-C₁₋₆-alkyl 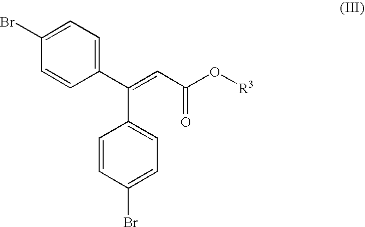
to an 3,3-bis-(4-bromophenyl)-prop-2-en-ol with formula IV 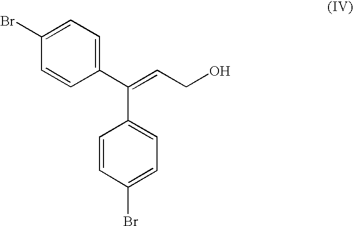
and
(b) reacting the compound of formula IV with
a halogenating agent to form the compound of formula I, wherein R is halogen, or
a reagent selected from the group consisting of SO₂R⁴ and R⁵SO₂R⁴, wherein R⁴ is halogen and R⁵ is selected from the group consisting of C₁₋₆-alkyl and C₁₋₆-alkyl-aryl, to form the compound of formula I, wherein R is OSO₂R¹.

4. The process according to claim 3, wherein the compound of formula III is a compound selected from the group consisting of
3,3-Bis-(4-bromophenyl)-acrylic acid methyl ester,
3,3-Bis-(4-bromophenyl)-acrylic acid ethyl ester,
3,3-Bis-(4-bromophenyl)-acrylic acid propyl ester,
3,3-Bis-(4-bromophenyl)-acrylic acid isopropyl ester,
3,3-Bis-(4-bromophenyl)-acrylic acid n-butyl ester,
3,3-Bis-(4-bromophenyl)-acrylic acid sec-butyl ester,
3,3-Bis-(4-bromophenyl)-acrylic acid tert-butyl ester, and
3,3-Bis-(4-bromophenyl)-acrylic acid benzyl ester.

5. The process according to claim 3, wherein the halogenating agent is SO(R⁴)₂ wherein R⁴ is halogen.

6. The process according to claim 3, wherein the reagent is selected from the group consisting of SO₂R⁴ and R⁵SO₂R⁴, wherein R⁴ is halogen and R⁵ is C₁₋₆-alkyl or C₁₋₆-alkyl-aryl.

7. The process according to claim 3, wherein the temperature is in the interval of 5-80° C.

8. The process according to claim 3, wherein the solvent in step (a) and/or step (b) is selected from the group consisting of toluene, N-methylpyrrolidinone (NMP), dimethyl formamide (DMF), and dimethyl acetamide (DMA).

9. The process according to claim 3, wherein the solvent is toluene in step (a).

10. The process according to claim 3, wherein the solvent is toluene in step (a) and step (b).

11. The process according to claim 3, wherein the compound obtained in step (a) is telescoped into step (b) after an aqueous wash.

12. A process for preparing a compound of formula II 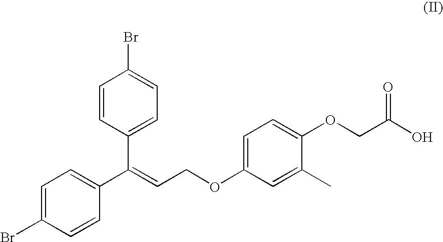
comprising the steps of
(a1) reacting a compound of formula I 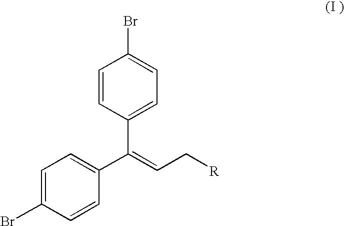
wherein R is selected from the group consisting of halogen and OSO₂R¹, wherein R¹ is C₁₋₆-alkyl or C₁₋₆-alkyl-aryl,
with a compound of formula V, wherein R² is C₁₋₆-alkyl 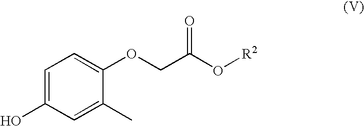
by an aliphatic nucleophilic substitution to form the ester of the compound of formula II and
(b1) hydrolysing said ester, optionally without isolation, to obtain the compound with formula II.

13. The process according to claim 12, wherein the aliphatic nucleophilic substitution is performed by phase transfer.

14. A process for preparing a compound of formula V, wherein R² is C₁₋₆-alkyl 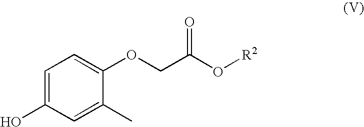
comprising the steps of
(a2) oxidising the compound with formula VI, wherein R⁶ is a protecting group and R⁷ is selected from the group consisting of a C₁₋₆-alkyl and aryl-C₁₋₆-alkyl, by a Baeyer-Villiger oxidation using an oxidizing agent 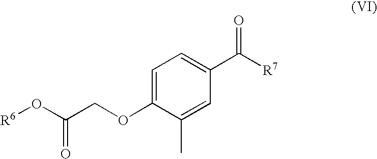
to a compound with formula VII 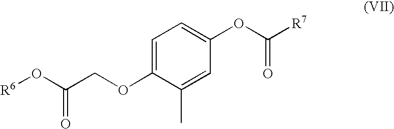
and
(b2) optionally without isolation of the compound of formula VII, deprotecting the compound of formula VII with a stoichiometric amount of sodium perborate hydrated to obtain the compound of formula V, and
(c2) isolating the compound of formula V by precipitation in an aqueous solution.

15. The process according to any claim 14, wherein the oxidizing agent used in Baeyer-Villiger oxidation is selected from the group consisting of peroxoacids, such as meta-chloroperoxybenzoic acid (m-CPBA), sodium perborate hydrated (such as sodium perborate monohydrate or sodium perborate tetrahydrate), urea-hydrogen peroxide complex, anhydrous hydrogen peroxide, peroxyacetic acid, and peroxytrifluoroacetic acid.

16. The process according to claim 15, wherein the oxidizing agent is sodium perborate hydrated.

17. The process according to claim 14, wherein the solvent in step (b2) is an alcohol or a mixture of an alcohol or toluene.
